(19) United States
(12) Patent Application Publication
(10) Pub. No.: US 2026/0117319 A1
(43) Pub. Date: Apr. 30, 2026

(54) ORAL MICROBIOME TESTING SYSTEM AND METHOD FOR EARLY DIAGNOSIS OF PERIODONTAL DISEASE IN PETS

(71) Applicant: REBIOGEN CO.,LTD, Gyeonggi-do (KR)

(72) Inventors: Junhyung LEE, Seoul (KR); Seonkyun KIM, Gyeonggi-do (KR)

(21) Appl. No.: 19/380,986

(22) Filed: Nov. 6, 2025

Related U.S. Application Data
(63) Continuation of application No. PCT/KR2025/013532, filed on Sep. 3, 2025.

(30) Foreign Application Priority Data
Nov. 29, 2024 (KR) 10-2024-0175241
Oct. 31, 2024 (KR) 10-2024-0152494

(51) Int. Cl. C12Q 1/689

(52) U.S. Cl. CPC C12Q 1/689; C12Q 2600/112

(57) ABSTRACT

According to some embodiments of the present invention, an oral microbiome testing system for early diagnosis of periodontal disease in a pet includes a genomic analysis device configured to generate microbiome data based on a specimen collected from the oral cavity of a pet prepared to be tested; and a processing device configured to generate abundance data, prevalence data, and correlation data of the microbiome data based on a list of microorganisms of interest in a pet standard database, generate health status data of the specimen by comparing the abundance data, prevalence data and correlation data with standard samples of the pet standard database, and generate an oral health report for the pet prepared to be tested based on the health status data.

### CROSS-REFERENCE TO RELATED APPLICATION

This application is a U.S. Bypass Continuation Application of International Application No. PCT/KR2025/013532, filed on September 03, 2025, which claims priority to and the benefit of Korean Patent Application No. 10-2024-0152494, filed on October 31, 2024, and Korean Patent Application No. 10-2024-0175241, filed on November 29, 2024, the disclosure of which is incorporated herein by reference in its entirety.

### BACKGROUND

### Technical Field

The present invention relates to a system and method of analyzing the oral microbiome of a pet for early diagnosis of an oral health status, such as periodontal disease.

### Background Art

Previously, the oral health status of a pet has been mainly evaluated through visual examinations, dental X-rays, and direct oral examination by a veterinarian, and it was determined through external signs such as dental condition, inflammation of the gums, and bad breath. Mechanical methods such as scaling were the main methods of managing an oral biofilm in pets, and a method of physically removing tartar using a dental tool or reducing biofilms through tooth cleaning by veterinarians was also utilized. Sometimes, a method of inhibiting bacterial growth in the oral cavity using an antibacterial agent or mouthwash was also used, making it difficult to control the balance of the microbiota.

Existing technology has had problems and improvements in various aspects. Visual examination and a physical method were effective in detecting an advanced disease, but there was a limitation in detecting changes in microbiota or a microscopic imbalance in the early stages of the disease. Existing microbial analysis techniques were useful for identifying the presence of microorganisms, but it was difficult to directly utilize them for prediction of health status. The mechanical removal method of managing a biofilm caused pain and stress during scaling, had a risk with general anesthesia, and was insufficient in that it was expensive and cumbersome because it required regular procedures. The use of antibacterials or detergents is effective for short-term bacterial reduction, but it was difficult to use it to regulate the long-term balance of the oral microbiota or to protect beneficial bacteria.

Studies on the oral microbiome of pets have been relatively few compared to that of humans, and there has been a lack of predictive models reflecting species-specific differences. It has been difficult to carefully assess the oral health status of an individual pet with conventional techniques. Particularly, the application of oral microbiome data to health prediction and customized prevention and care has lacked a standardized method of interpreting analytical results clinically.

### RELATED ART DOCUMENTS

Patent Document 1: Korean Laid-Open Patent Publication No. 10-2023-0173676 (Dec. 27, 2023)

Patent Document 2: Korean Laid-Open Patent Publication No. 10-2024-0033007 (Mar. 12, 2024)

### SUMMARY

### Technical Problem

One of objectives of the present invention is to provide a testing system and method that may diagnose the oral health status of a pet prepared to be tested by generating and analyzing microbiome data from a specimen collected from the oral cavity, to improve limitations of existing oral care methods for pets. The technical objectives of the present invention are not limited to the technical problems mentioned above, and other technical problems that are not mentioned above can be clearly understood by those of ordinary skill in the art from the following descriptions.

### Technical Solution

According to some embodiments of the present invention, an oral microbiome testing system for early diagnosis of periodontal disease in a pet may include: a genomic analysis device configured to generate microbiome data based on a specimen collected from the oral cavity of a pet prepared to be tested; and a processing device configured to generate abundance data, prevalence data, and correlation data of the microbiome data based on a list of microorganisms of interest in a pet standard database, generate health status data of the specimen by comparing the abundance data, prevalence data and correlation data with standard samples of the pet standard database, and generate an oral health report for the pet prepared to be tested based on the health status data.

According to some embodiments of the present invention, the genomic analysis device may be configured to generate the microbiome data by identifying types of microorganisms present in the specimen through 16S rRNA sequencing based on next generation sequencing (NGS).

According to some embodiments of the present invention, the list of microorganisms of interest may be configured to define n phyla and m genera referred to for the early diagnosis of periodontal disease, and the processing device may be configured to generate the abundance data, prevalence data and correlation data based on the n phyla and the m genera.

According to some embodiments of the present invention, the processing device may be configured to generate the abundance data by calculating a first compositional ratio of microorganisms present in the specimen across the n phyla and a second compositional ratio of microorganisms present in the specimen across the m genera, and generate the health status data of the specimen by comparing the first compositional ratio and the second compositional ratio with the standard samples.

According to some embodiments of the present invention, the processing device may be configured to generate the prevalence data by generating a first heatmap representing the probabilities that each of the n phyla will exceed a detection threshold of relative abundance and a second heatmap representing the probabilities that each of the m genera will exceed a detection threshold of relative abundance, and generate the health status data of the specimen by comparing the first heatmap and the second heatmap with the standard samples.

According to some embodiments of the present invention, the processing device may be configured to generate the correlation data by calculating m correlation values between m microbial populations and the representative microbial population, present in the specimen, as compared with the m genera, and to generate health status data of the specimen by comparing the m correlation values with the standard samples.

According to some embodiments of the present invention, the processing device may be configured to determine a standard sample closest to the abundance data, prevalence data and correlation data for the specimen using a data classification model that is trained to output the standard sample closest to the input data among the standard samples, and to generate the health status data based on the standard sample determined to be closest to the specimen.

According to some embodiments of the present invention, the processing device may be configured to generate the oral health report by considering metadata including breed, age, sex, body weight, oral care status, and specific diseases of the pet prepared to be tested.

According to some embodiments of the present invention, the standard samples of the pet standard database may be constructed based on five disease stages consisting of bad breath, plaque, tartar, gum inflammation, and periodontal disease, and five progression stages rated with scores of 1, 2, 3, 4, and 5 for each of the five disease stages.

According to some embodiments of the present invention, the processing device may be configured to determine an evaluation indicator corresponding to the microbiome data based on the PCoA cluster of the pet standard database, and generate the oral health report in further consideration of the evaluation indicator.

According to some embodiments of the present invention, the oral microbiome testing method for early diagnosis of periodontal disease in pets may include: generating microbiome data based on a specimen collected from the oral cavity of a pet prepared to be tested using a genomic analysis device; generating abundance data, prevalence data and correlation data of the microbiome data based on a list of microorganisms of interest in a pet standard database; generating health status data of the specimen by comparing the abundance data, prevalence data and correlation data with standard samples of the pet standard database using the processing device; and generating an oral health report for the pet prepared to be tested based on the health status data using the processing device.

### Advantageous Effects

According to embodiments of the present invention, in order to improve the limitations of the existing pet oral care methods, a testing system and method that can diagnose the oral health status of a pet prepared to be tested by generating microbiome data from a specimen collected from the oral cavity and analyzing the data can be provided.

Technical effects according to embodiments of the present invention are not limited to those mentioned above, and other effects that are not mentioned will be clearly understood by those of ordinary skill in the art in accordance with the disclosure of the present disclosure.

### BRIEF DESCRIPTION OF THE DRAWINGS

FIG. 1 illustrates an environment where an oral microbiome testing system for early diagnosis of periodontal disease in a pet operates according to some embodiments.

FIG. 2 illustrates components that constitute an oral microbiome testing system for early diagnosis of periodontal disease in pets according to some embodiments.

FIG. 3 illustrates a process of generating an oral health report based on a specimen collected from the oral cavity by an oral microbiome testing system according to some embodiments.

FIG. 4 illustrates a method of collecting a specimen from the oral cavity of a pet prepared to be tested according to some embodiments.

FIG. 5 illustrates a method of manufacturing and optimizing a primer and a probe for biomarkers according to some embodiments.

FIG. 6 illustrates abundance data according to some embodiments.

FIG. 7 illustrates prevalence data according to some embodiments.

FIGS. 8 and 9 illustrate correlation data according to some embodiments.

FIG. 10 illustrates a method of classifying the status of a specimen based on the PCoA cluster for the representative microorganism according to some embodiments.

FIG. 11 illustrates metadata further considered to generate health status data of a pet prepared to be tested according to some embodiments.

FIG. 12 illustrates operations that constitute the oral microbiome testing process for early diagnosis of periodontal disease in a pet.

### DETAILED DESCRIPTION

An oral microbiome testing system for early diagnosis of periodontal disease in a pet includes a genomic analysis device configured to generate microbiome data based on a specimen collected from the oral cavity of a pet prepared to be tested; and a processing device configured to generate abundance data, prevalence data, and correlation data of the microbiome data based on the list of microorganisms of interest in the pet standard database, generate the health status data of the specimen by comparing the abundance data, prevalence data and correlation data with standard samples of the pet standard database, and generate an oral health report for the pet prepared to be tested based on the health status data. The standard samples of the pet standard database are constructed with primary standard samples including stages of abundance data, prevalence data, and correlation data for a plurality of conditions including bad breath, plaque, tartar, gum inflammation, and periodontal disease, and additional secondary standard samples including abundance data, prevalence data, and correlation data for each stage of disease progression by subdividing the disease progression stages for the primary standard samples. The prevalence data is provided as a heatmap that visually indicates the appearance frequency of each microorganism by displaying, in different colors, the probability that a specific microorganism in the sample exceeds a predetermined threshold in the primary and secondary standard samples.

Hereinafter, embodiments of the present invention will be described in detail with reference to the drawings. The following description is merely provided to specify the embodiments, and is not intended to limit the scope of the present invention. That which one of ordinary skill in the art can readily infer from the detailed description and embodiments of the present invention should be construed as within the scope of the present invention. Detailed description of matters well known to those of ordinary skill in the art of the present invention will be omitted.

Although the terminology used in the present invention is described using general terms widely used in the art, the meanings of the terminology used in the present invention may vary depending on the intention of a technician in the field, the emergence of a new technology, the criteria for examination, or precedent. Some terms may be arbitrarily selected by the applicant, and in this case, the meanings of the arbitrarily selected terms will be described in detail. The terms used in the present invention should be construed in a sense that reflects the overall context of the specification, not just the dictionary meaning.

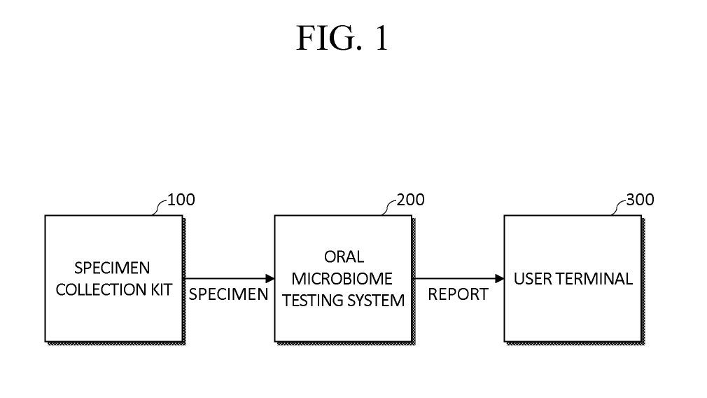

FIG. 1 illustrates an environment where an oral microbiome testing system for early diagnosis of periodontal disease in pets operates according to some embodiments.

Referring to FIG. 1, an oral microbiome testing system 200 for early diagnosis of periodontal disease in a pet may generate an oral health report for a pet prepared to be tested from a specimen collected by a specimen collection kit 100 and provide the report to a user terminal 300.

A user may collect a specimen from the oral cavity of the tested pet using the specimen collection kit 100. The oral microbiome testing system 200 performs genomic analysis on the specimen and processes the resulting data to generate health status data of the tested pet, thereby generating oral health report based on this. The user may view the oral health report using the user terminal 300. The environment where the oral microbiome testing system 200 operates may be embodied using a computer program and/or mobile application, and the user terminal 300 may be a user’s mobile phone and/or PC.

The oral microbiome testing system 200 may have the purpose of overcoming the limitations of existing technology and contributing to the overall health promotion of the pet by precisely analyzing the oral microorganism data of the pet and utilizing the data for preventive health care. The correlation, prevalence and abundance data of the microorganisms harvested from the oral cavity are analyzed to capture an early-stage disease signal and enable early response to the signal.

Through the learning of microbial patterns, beneficial bacteria can be protected and only pathogenic bacteria can be effectively managed to maintain the balance of the microbiota. Efficiency can be enhanced by using data-based prediction models to present prevention and care methods suitable for the characteristics of an individual pet. By systemically analyzing the oral microorganism data of the pet and taking preventive measures, benefits such as disease prevention, health promotion, cost reduction, and improvement of the quality of life of the pet through customized care can be expected.

The oral microbiome testing system 200 may analyze the microbiome in the oral cavity of the pet by utilizing NGS, and based on the results of analyzing the correlation, prevalence and abundance data of the microorganisms, disease signals may be detected, enabling early response in early stages.

In collaboration with veterinary hospitals, approximately 500 or more oral samples may be collected, and 16S rRNA sequencing may be performed using genomic sequencing (e.g., Illumina MiSeq) for these samples.

In exemplary embodiments, the correlation, prevalence and abundance data of the microorganisms present in the oral cavity and their pattern changes may be identified in each of bad breath, plaque, tartar, gum inflammation, and periodontal disease. A domestic pet standard database (DB) can be constructed using oral samples, through which the microbiome (entire microbial populations) may be analyzed.

The 16S rRNA sequencing technique may refer to a technique used to analyze a specific gene (16S rRNA) of bacteria or microorganisms to determine what types of bacteria/microorganisms are present and to distinguish between different bacteria. Since a different type of bacteria has different genes, it may be possible to quickly and accurately identify the bacterial type through the 16S rRNA sequencing technique. 16S rRNA is present only in bacteria and archaea, and therefore the type and amount of all bacteria present in the specimen may be identified.

Microbiome data may be generated by analyzing bacteria/microorganisms present in specimens collected from the oral cavity of dogs or the like, from which correlation data, prevalence data, and abundance data may be derived. The oral health status of the pet may be determined based on the above-mentioned data.

The oral microbiome testing system 200 may acquire data according to the stage of progression including bad breath (initial disease), plaque, gum inflammation, tartar, and periodontal disease. Such data may be acquired from the oral samples of pets with diseases according to respective stages of progression at a veterinary hospital. Veterinary hospital data for the construction of a standard DB may further include metadata such as species, age, sex including neuter status (F, M, CM, IF), extraction status, scaling status, and any specific diseases for the construction of a standard database.

The oral microbiome testing system 200 may utilize NGS data to read interactions between all microorganisms in each specimen. Beyond the interaction of some microorganisms of one or two species, it may be possible to determine the types and trends between approximately 10⁷ or more bacteria/microorganisms present in the oral cavity, which may be classified at the phylum and genus levels. At the phylum and genus levels, the top m/n bacterial/microbial types with a high correlation with the pet oral status may be selected, and the pet oral health may be diagnosed using these.

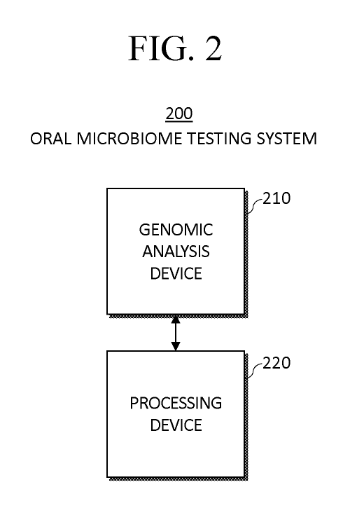

FIG. 2 illustrates components that constitute an oral microbiome testing system for early diagnosis of periodontal disease in a pet according to some embodiments.

Referring to FIG. 2, an oral microbiome testing system 200 for early diagnosis of periodontal disease in a pet may include a genomic analysis device 210 and a processing device 220. However, there is no limitation on the constituent components of the system, and some components may be omitted from the oral microbiome testing system 200, or other general-purpose components may be further included in the oral microbiome testing system 200.

The genomic analysis device 210 may include a PCR device capable of performing genomic analysis on specimens collected from the specimen collection kit 100. For example, the genomic analysis device 210 may be a third-generation PCR device and/or NGS device.

The genomic analysis device 210 may identify the types of bacteria/microorganisms in each specimen to generate microbiome data or oral microbiome data in the oral cavity. The type and amount of bacteria/microorganisms present in the pet oral cavity may be analyzed based on the oral microbiome data.

The processing device 220 may perform processing procedures on the oral microbiome data analyzed by the genomic analysis device 210 to generate health status data and an oral health report. The processing device 220 may include a memory and a processor. The memory may store various data, instructions, computer programs, software, mobile applications, etc. The processor can execute control operations by executing instructions or the like, stored in the memory. For example, the memory may include a non-volatile memory, a volatile memory, a storage device, etc., and the processor may include a microprocessor, CPU, GPU, AP, etc.

The genomic analysis device 210 may be configured to generate microbiome data based on a specimen collected from the oral cavity of the tested pet. The genomic analysis device 210 may identify all bacteria/microorganisms present in the specimen through three-dimensional PCR and/or NGS sequencing to generate the microbiome data.

The processing device 220 may be configured to generate the abundance data, prevalence data, and correlation data of the microbiome data based on the list of microorganisms of interest in the pet standard database. The pet standard database may be constructed based on oral status-specific data provided through a veterinary hospital, etc.

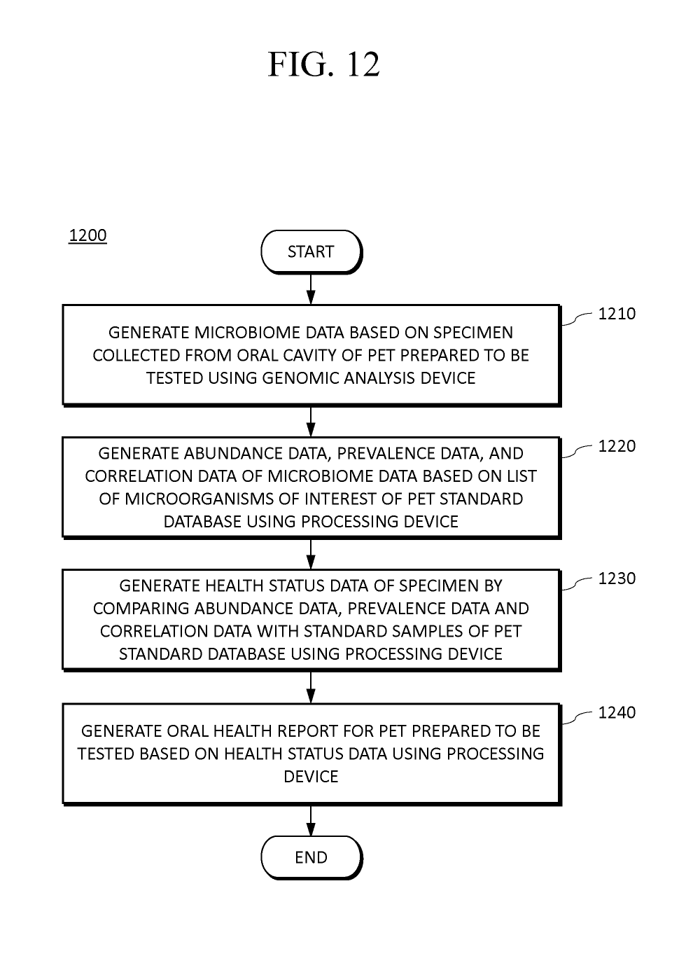

The oral microbiome testing system 200 may include an output device. In an exemplary embodiment, the output device may include a printer. As will be described in FIG. 12, the processing device may generate an oral health report for a pet prepared to be tested based on the health status data, and deliver an output signal to the printer. The printer may receive the output signal and then output the health report.

That is, the processing device may generate microbiome data based on an oral specimen, generate abundance data, prevalence data, and correlation data based on the microbiome data, generate health status data of a pet based on them, and generate an oral health report based on the health status data. In addition, the processing device may automatically deliver the output signal to the printer when the generation of the oral health report is completed, and the printer receiving the output signal may automatically output the oral health report.

The pet standard database may have the bacterial/microbial lists applied to diagnose the oral status of the pet. Microorganisms of interest in the list may vary in detection depending on the oral status of the pet, and the top n/m species of bacteria/microorganisms best suitable for diagnosis of oral conditions may be registered in the list of microorganisms of interest.

Abundance data may indicate the detection ratio of n/m species of bacteria/microorganisms. The prevalence data may represent the frequency/probability of detection of n/m species of bacteria/microorganisms in the form of a two-dimensional heatmap. The correlation data may represent the correlation coefficient values between the microorganisms in the microbial list of interest and the representative microorganism.

The processing device 220 may be configured to generate the health status data of specimens by comparing the standard samples of the pet standard database based on at least one of the abundance data, prevalence data, and correlation data. For example, the processing device 220 may be configured to generate the health status data of specimens by comparing the standard samples of the pet standard database based on at least one of the abundance data, prevalence data, and correlation data.

The pet standard database may have standard samples generated based on data collected by oral status at a veterinary hospital or the like. The standard samples may classify oral diseases into stages including bad breath, plaque and tartar, and each stage may be further classified into five levels of severity.

For example, the standard samples may include abundance data, prevalence data and correlation data for a specific oral disease status and specific severity. The data of the specimen may be compared with the standard samples, and the standard sample closest to the data of the specimen may be considered as indicative of the condition of the pet prepared to be tested.

The selection of the closest data may be performed through a machine learning-based AI model and/or a statistical technique. When a standard sample closest to the specimen data is selected, the health status data of the specimen may be generated based on this.

The processing device 220 may be configured to generate an oral health report for a pet prepared to be tested based on the health status data. The health status data may indicate whether the current oral condition of the pet prepared to be tested corresponds to any of a bad breath stage, a plaque stage, and a tartar stage, and the severity of the corresponding stage. Collectively, it may also indicate whether the condition for the pet prepared to be tested is good, moderate, or poor.

For example, the health status data may include the cause of occurrence, a symptom or sign, a preventive method, and a care method. The oral health report may include solutions for the oral health status. For example, when the pet is a dog, the oral health report may provide a customized solution based on metadata such as dog breed, age, gender, and an underlying disease. The oral health report may be provided to a user terminal 300 using an application/program.

According to one embodiment, the genomic analysis device 210 may be configured to generate microbiome data by identifying the type of a microorganism present in a specimen by performing 16S rRNA sequencing based on NGS. The NGS is a genomic analysis technique capable of rapidly sequencing a large amount of DNA or RNA, allowing millions of small DNA fragments to be sequenced simultaneously, unlike conventional sequencing techniques. For example, the NGS may be performed based on a three-dimensional PCR technique. The types of microorganisms present in the specimen may be identified through 16S rRNA sequencing.

According to one embodiment, the microbial list of interest may define n phyla and m genera referenced for early diagnosis of periodontal disease, and the processing device 220 may be configured to generate abundance data, prevalence data and correlation data based on the n phyla and m genera.

Generally, there are a large number of microorganisms present in the oral specimen of a pet, but the microorganisms utilized in the diagnosis of oral status may be limited. Meanwhile, the classification of the microorganisms may be performed on the basis of phylum, or on the basis of genus in other units. It may be possible to analyze the microorganisms at different dimensions by considering phylum and genus together.

According to one embodiment, the processing device 220 may be configured to generate abundance data by calculating a first compositional ratio for n phyla of microorganisms and a second compositional ratio for m genera, present in the specimen, and to generate the health status data of the specimen by comparing the first and second compositional ratios with the standard samples.

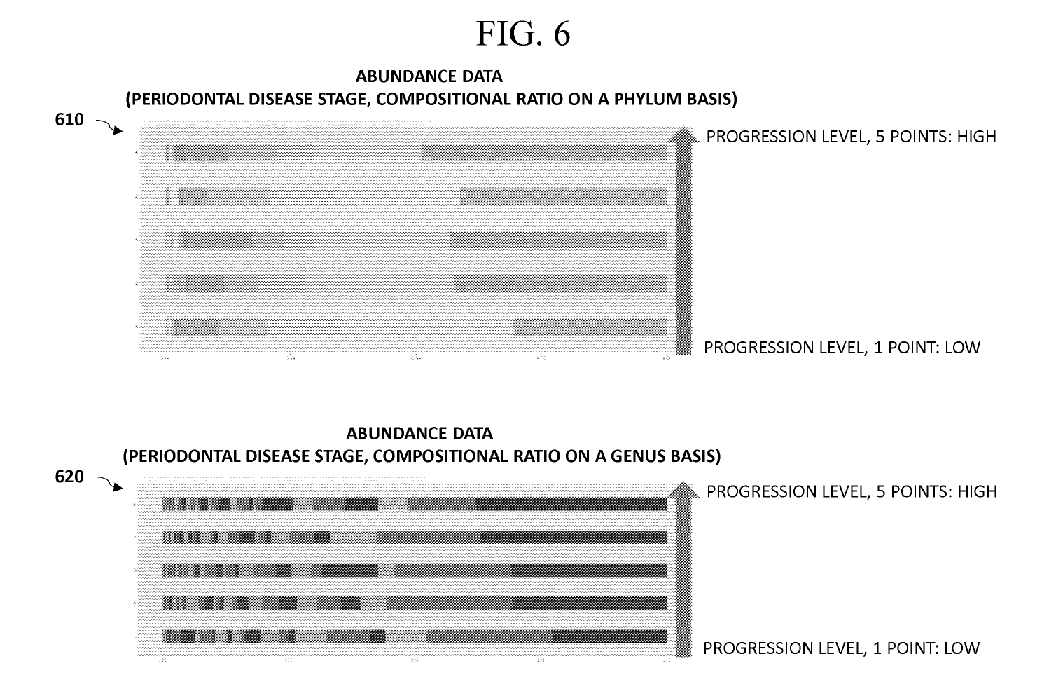

The first compositional ratio 610 and the second compositional ratio 620 can be understood with reference to FIG. 6 later. The oral status of the pet may be diagnosed depending on which standard sample is closest to the compositional ratios generated for the specimen.

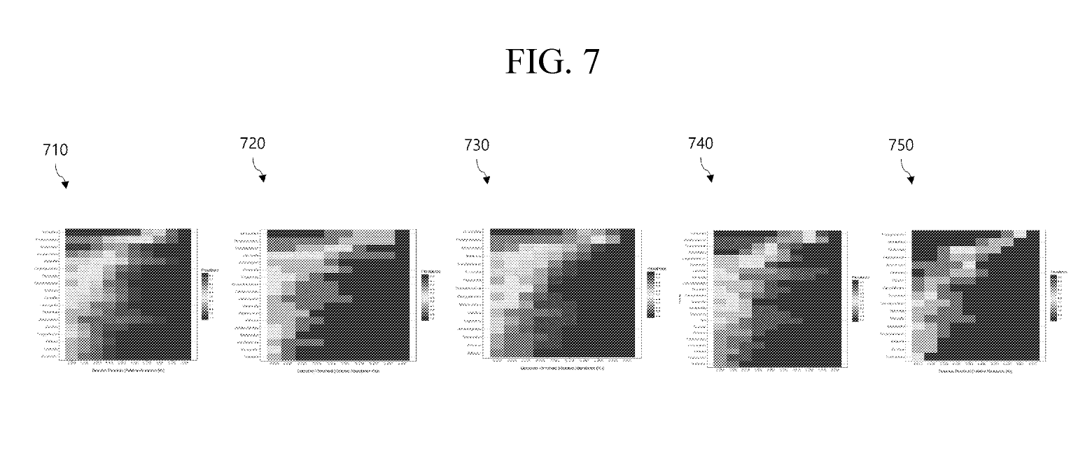

According to one embodiment, the processing device 220 may be configured to generate prevalence data by generating a first heatmap representing the probabilities that each of the n phyla exceeds its relative abundance of detection threshold and a second heatmap representing the probabilities that each of the m genera exceeds its relative abundance of detection threshold, and to generate the health status data of a specimen by comparing the first heatmap and the second heatmap with the standard samples. The first and second heatmaps can be understood with reference to FIG. 7 below. The heatmaps may visually display the occurrence frequency of each microorganism by displaying, in different colors, the probabilities that specific microorganisms exceed specific thresholds. Standard samples for each of the first and second heatmaps may be generated by progression degree/severity on a 1-to-5 scale.

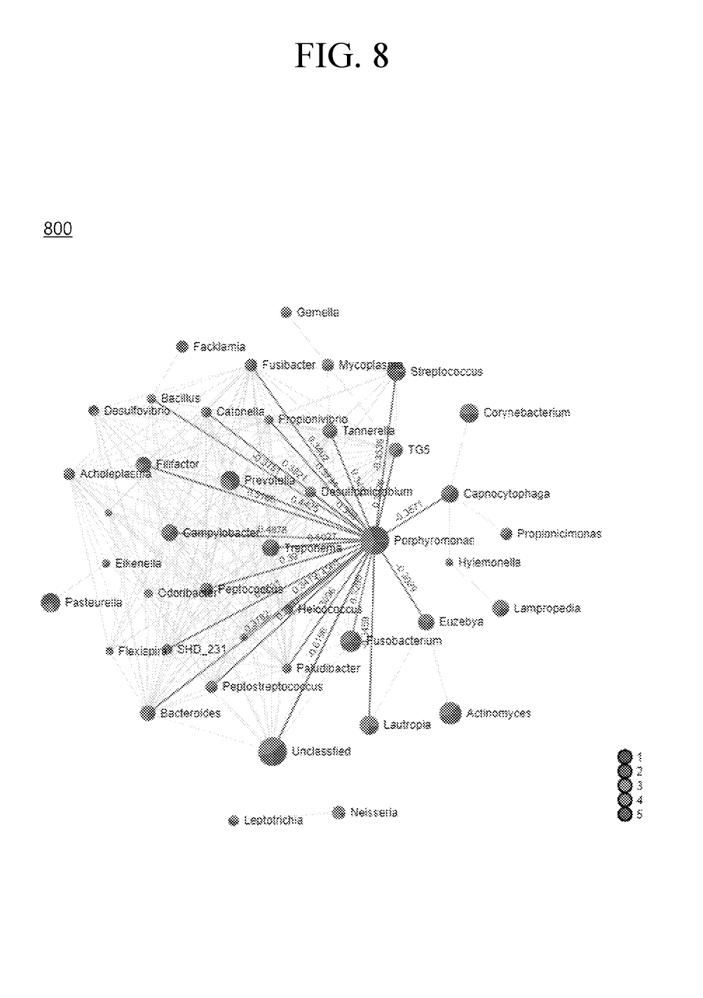

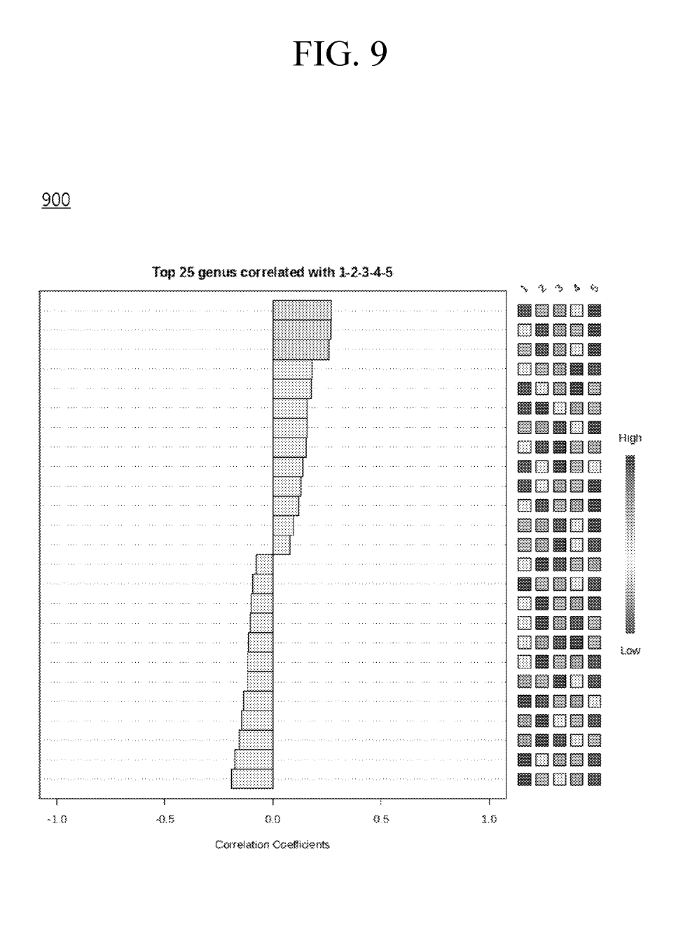

According to one embodiment, the processing device 220 may be configured to generate correlation data by calculating m correlation values between m microbial populations present in the specimen and the representative microbial population, corresponding to m genera, and to generate health status data of the specimen by comparing the m correlation values with standard samples. A representative microorganism may be selected as a microorganism that is the best indicator of the oral condition of a pet. Based on this, the top m types of highly correlated microorganisms may be registered in a pet standard database, and for the specimen to be diagnosed, m correlation values between the m genera and the representative microorganism may be generated. The m correlation values may be utilized as an indicator for diagnosing the oral status of a pet to be diagnosed. For example, the m correlation values may be utilized as input data of a model trained to diagnose an oral status. The correlation data can be understood with reference to FIGS. 8 and 9 below.

According to one embodiment, the processing device 220 may be configured to determine a standard sample closest to the abundance data, prevalence data and correlation data for a specimen using a data classification model trained to output a standard sample closest to input data among the standard samples, and generate health status data based on a standard sample determined to be closest to the specimen.

For example, three standard samples closest to each of abundance data, prevalence data, and correlation data may be selected, and health status data may be generated based on the three standard samples. Each standard sample may indicate which disease stage it belongs to among bad breath, plaque, tartar, gum inflammation, and periodontal disease, as well as the severity level of each disease stage on a scale from 1 to 5.

According to one embodiment, the processing device 220 may be configured to generate an oral health report by considering metadata including breed, age, sex, body weight, oral care status, and specific diseases of the pet prepared to be tested. The oral health report may be generated based on the health status data and metadata. The metadata may be referenced to provide an optimized solution for each pet prepared to be tested.

According to one embodiment, the standard samples of the pet standard database may be constructed based on five disease stages consisting of bad breath, plaque, tartar, gum inflammation, and periodontal disease, and five progression stages rated with scores of 1, 2, 3, 4, and 5 for each of the five disease stages.

For example, the standard samples for abundance data may be prepared in advance for 25 cases corresponding to the combinations of the five disease stages and the five progression stages as will be described in FIG. 6, and prevalence data and correlation data may also be prepared in a similar way.

According to one embodiment, the processing device 220 may be configured to determine evaluation indicators corresponding to microbiome data based on the PCoA cluster of the pet standard database, and generate an oral health report by further considering the evaluation indicators.

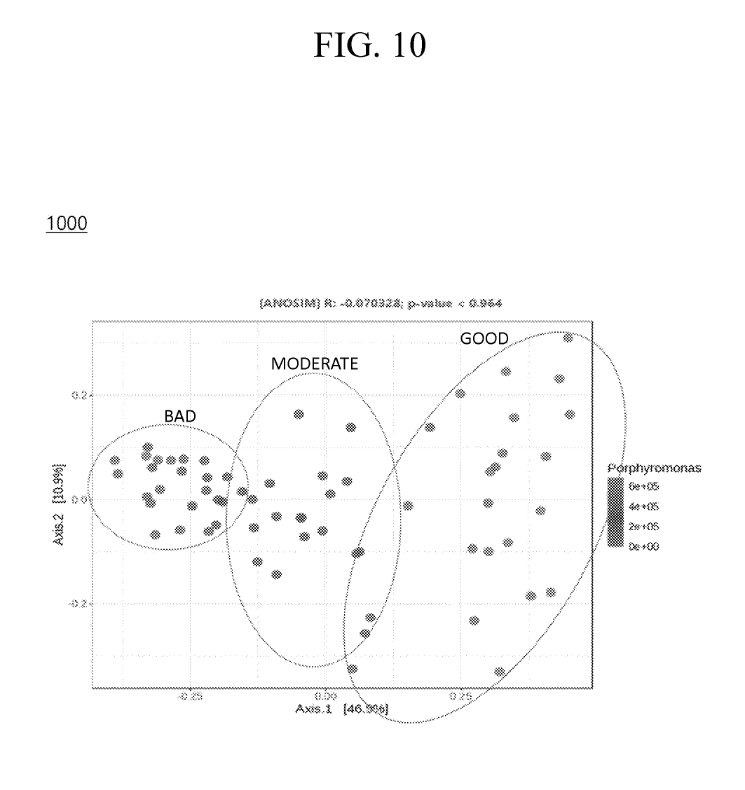

For example, as will be shown in FIG. 10 below, the PCoA cluster may be classified into bad/moderate/good, and such classification may be performed based on a representative microorganism that serves as the reference for generating correlation data. That is, the evaluation indicators corresponding to the microbiome data based on the detection level of the representative microorganism present in a specimen from a pet prepared to be tested, and information thereon may be included in the oral health report.

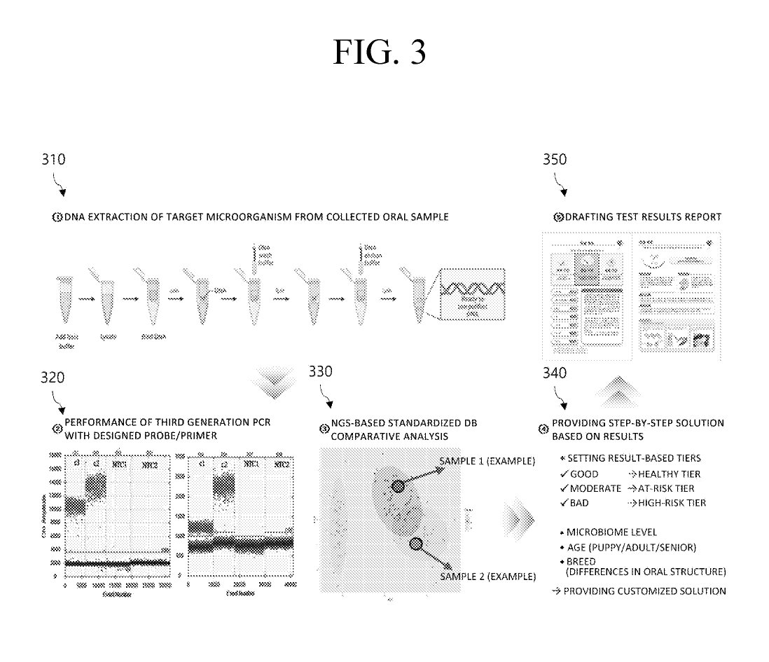

FIG. 3 illustrates a process of generating an oral health report based on a specimen collected from the oral cavity by an oral microbiome testing system according to some embodiments.

Referring to FIG. 3, processes 310 to 350 of generating an oral health report based on a specimen collected from the oral cavity by the oral microbiome testing system can be seen.

In the first and second processes 310 and 320, a genomic analysis device 210 may extract DNA by performing genomic analysis on the specimen collected from the oral cavity of a pet prepared to be tested, and generate microbiome data. For example, the microbiome data may be generated using NGS and/or 3-dimensional PCR-based 16S rRNA sequencing.

In the third process 330, the microbiome data generated from the specimen may be compared with pet standard database. The pet standard database may have multiple standard samples, and the standard samples closest to the specimen data may be selected. For example, the abundance, prevalence, and correlation of the specimen data may be compared with the standard samples, and the standard samples closest to the specimen data may be selected using a statistical technique or AI model trained based on machine learning.

In the fourth process 340, health status data may be generated based on the results of the comparison with the standard samples, and a step-by-step solution may be generated based on this. In the fifth process 350, an oral health report for a pet prepared to be tested may be generated based on a step-by-step solution, which may be provided to a user using a user terminal 300.

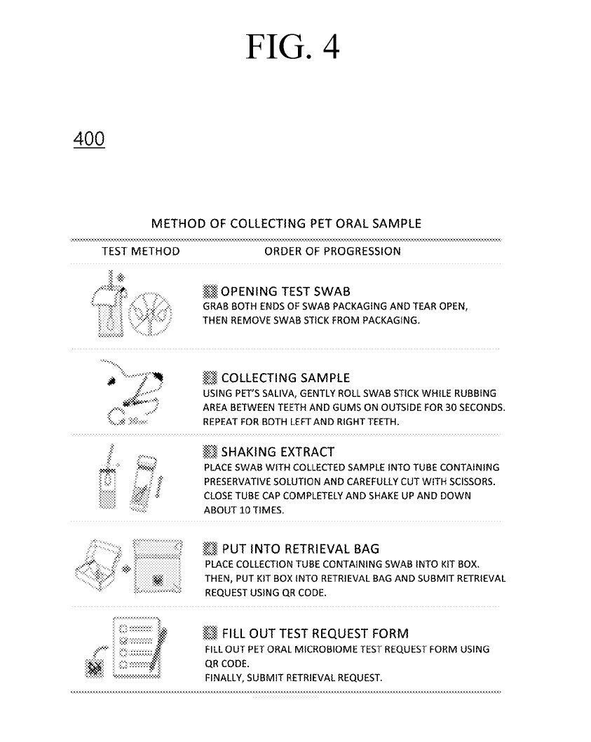

FIG. 4 illustrates a method of collecting a specimen from the oral cavity of a pet prepared to be tested according to some embodiments.

Referring to FIG. 4, a manual 400 illustrating a method of collecting a specimen from the oral cavity of a pet prepared to be tested may be shown.

According to the manual 400, a method of using the specimen collection kit 100 may consist of five operations, including: opening a test swab, collecting a sample, shaking an extract, collecting the resulting extract in a retrieval bag, and submitting a test request. When a specimen of the pet prepared to be tested is collected according to the manual 400, the specimen may be delivered to an oral microbiome testing system 200 for genomic analysis.

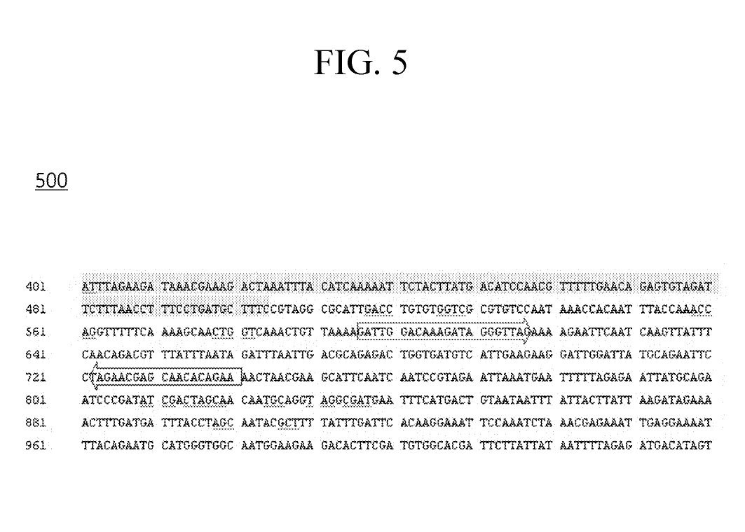

FIG. 5 illustrates a method of manufacturing and optimizing a primer and a probe for biomarkers according to some embodiments.

Referring to FIG. 5, an image 500 illustrating a method of manufacturing a primer and a probe for biomarkers and optimizing them may be shown.

The image 500 may visualize a tool for manufacturing a primer and/or a probe for detecting the selected n/m microorganisms of interest and optimizing them. Each of the selected n/m microorganisms of interest may be a biomarker. For each biomarker, two primers and two probes may be manufactured. When the manufacture and optimization of the primers/probes for the microorganisms of interest are completed, microbiome data may be analyzed from the specimen using them.

For primer manufacture and optimization, various items may be considered. For example, primer Tm values should be similar to ±2 ℃, and for 5' nucleic acid qPCR analysis, may be generally near 60 to 62 ℃, may target 18 to 30 bases, and may not continuously include 4 or more Gs. The GC content should range from 35 to 65%. Similarly, for probe manufacture and optimization, the Tm values should be 4 to 10 ℃ higher than the primer, G should not be continuous, the content of G+C should be 30 to 80%, G may not come at the 5' end, the probe length should not be greater than 30 bases, and the probe may not be designed as a sense or antisense strand.

FIG. 6 illustrates abundance data according to some embodiments.

Referring to FIG. 6, a first compositional ratio 610 and a second compositional ratio 620 corresponding to abundance data may be illustrated. The first compositional ratio 610 and the second compositional ratio 620 may be registered in the pet standard database.

Abundance data, which is data representing a relative proportion of the microorganisms as a percentage, may be secured. Abundance data according to each of the five conditions such as bad breath, plaque tartar, gum inflammation, and periodontal disease may be collected. From this data, the abundance (proportion by type) of microorganisms according to the oral status may be obtained. As shown in this graph, data may be obtained by setting the data categories differently, such as phylum and genus levels.

The first compositional ratio 610 and the second compositional ratio 620 may be standard samples for the periodontal disease stage, and in addition, standard samples in the forms of the first compositional ratio 610 and the second compositional ratio 620 for bad breath, plaque, tartar, gum inflammation, and periodontal disease may be included in a DB.

The first compositional ratio 610 represents the proportion of microorganisms of interest that are classified based on the phylum, while the second compositional ratio 620 may represent the proportion of microorganisms of interest that are classified based on the genus. Since it is possible to analyze the microorganisms of interest based on a different scale, the microbiome data may be analyzed more diversely.

The first compositional ratio 610 and the second compositional ratio 620 may indicate the standard samples for the five-level severity rated with scores of 1 to 5. The severity stage of the specimen may be determined based on which sample the distribution of abundance data of microorganisms present in the specimen is closest to.

FIG. 7 illustrates prevalence data according to some embodiments.

Referring to FIG. 7, heatmap data 710 to 750 corresponding to the prevalence data may be illustrated. The heatmap data 710 to 750 may correspond to the five-level severity rated with scores of 1 to 5, respectively. The heatmap data 710 to 750 may be generated for any one of the five stages of bad breath, plaque, tartar, gum inflammation, and periodontal disease, and may be generated based on the phylum or genus level.

The vertical axis of the heatmap data 710 to 750 may be microorganisms of interest classified at the phylum level, or microorganisms of interest classified at the genus level, and the horizontal axis of the heatmap data 710 to 750 may be a detection threshold for relative abundance. The colors of the heatmap data 710 to 750 may indicate the probability that the corresponding microorganisms will be detected in the specimen for the corresponding detection threshold. For example, when the color for the detection threshold of 0.010 of a particular microorganism is the darkest color, it can be interpreted that the probability of the detection of the corresponding microorganisms for the abundance of 0.010 is 100%.

The prevalence data expressed in the form of the heatmap data 710 to 750 may be an indicator to explain characteristics of the microbiome data of the specimen. The heatmap data for the combination of five stages of disease progression and severity rated on a scale of 1 to 5 points may be registered in a DB as standard samples, and the oral health status of the specimen may be analyzed in comparison with the specimen data. The comparison of the specimen data with the standard samples may be performed based on a machine learning-based AL model and/or a statistical technique.

FIGS. 8 and 9 illustrate correlation data according to some embodiments.

Referring to FIG. 8, a graph 800 illustrating correlation data may be shown. Each node of the graph 800 may be a microorganism of interest or a biomarker.

In the correlation, the connection line between nodes may be divided into a line representing positive correlation and a line representing negative correlation, which may be distinguished by color. The size of each node may indicate the frequency or importance of the microorganisms, and the correlation data may indicate the relationship between microorganisms.

For example, the correlation data may include correlation coefficient values between m microorganisms corresponding to the m genera of microorganisms of the list of microorganisms of interest, and the representative microorganism. Alternatively, the correlation data may be generated based on n phyla.

The graph 800 may be generated for each of the combinations of five stages of disease progression and severity rated on a scale of 1 to 5 points, which may be registered as the standard samples of a DB. The correlation data may be an indicator to explain characteristics of the microbiome data of the specimen, and the oral health status of the specimen data may be estimated by comparing the specimen data of a pet prepared to be tested and standard samples.

Referring to FIG. 9, a graph 900 showing correlation coefficient values between the representative microorganism and microorganisms of interest in a 25-genus unit may be shown. Since the microorganisms of interest in a 25-genus unit have a high correlation to the representative microorganism, the variation in detection level may be large depending on the oral status of a pet prepared to be tested. In consideration of this, the top 25 microorganisms may be selected as biomarkers or microorganisms of interest based on the correlation coefficient values, and may be registered in a DB to be utilized in the comparison with the specimen data.

FIG. 10 illustrates a method of classifying the status of a specimen based on the PCoA cluster for the representative microorganism according to some embodiments.

Referring to FIG. 10, a graph 1000 illustrating a method of classifying the state of a specimen based on the PCoA cluster for the representative microorganism may be shown.

The graph 1000 may represent a PCoA cluster for a particular type of representative microorganism. The PCoA cluster may be classified as good/moderate/bad through a data clustering technique, and such classification criteria may be registered in a DB. When new data on the representative microorganism is collected from a specimen of a pet prepared to be tested, it may be compared with the PCoA cluster of the DB to determine whether the new data belongs to good/moderate/bad, and a health status report can be generated based on this.

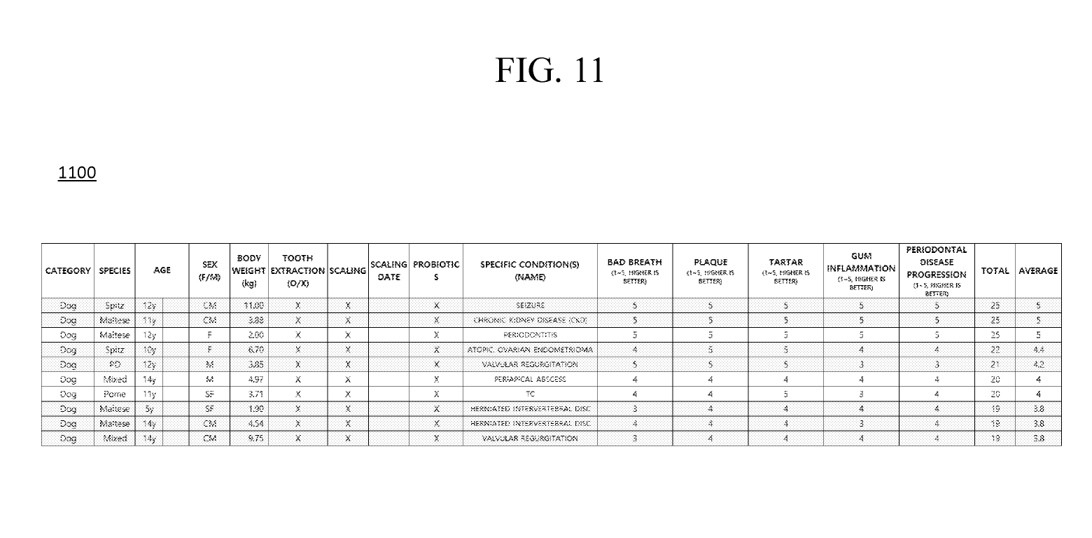

FIG. 11 illustrates metadata further considered to generate health status data of a pet prepared to be tested according to some embodiments.

Referring to FIG. 11, a table 1100 exemplifying metadata further considered to generate the health status data of a pet prepared to be tested may be shown.

The metadata exemplified in the table 1100 may include distinctions regarding a pet type, breed, age, sex (considering neuter status), body weight, history of tooth extraction, history of dental scaling, date of scaling, history of probiotic consumption, and history of a specific disease. For example, the data displayed in the table 1100 may be associated with the standard samples registered in a DB. The standard samples may be labeled with five stages of disease progression and severity on a scale of 1 to 5 points.

FIG. 12 illustrates operations that constitute the oral microbiome testing method for early diagnosis of periodontal disease in a pet.

Referring to FIG. 12, a method 1200 of testing the oral microbiome for early diagnosis of periodontal disease in a pet may include operation 1210 to 1240. However, there is no limitation on the method, and some operations may be omitted, or other general-purpose operations may be added. The operations of the method 1200 of testing the oral microbiome may be performed in a different order than that shown in FIG. 12.

The oral microbiome testing method 1200 may include operations that are time-series processed in the oral microbiome testing system 200. Accordingly, even if omitted below, the above description of the oral microbiome testing system 200 may be equally applicable to the oral microbiome testing method 1200.

Operations 1210 to 1240 of the oral microbiome testing method 1200 may be performed by a genomic analysis device 210 and a processing device 220 of the oral microbiome testing system 200.

In operation 1210, the oral microbiome testing system 200 may execute an operation of generating microbiome data based on a specimen collected from the oral cavity of a pet prepared to be tested using a genomic analysis device.

In operation 1220, the oral microbiome testing system 200 may execute a step of generating abundance data, prevalence data, and correlation data of the microbiome data based on the list of microorganisms of interest of a pet standard database using a processing device.

In operation 1230, the oral microbiome testing system 200 may execute an operation of generating health status data of a specimen by comparing the abundance data, prevalence data and correlation data with standard samples of the pet standard database using the processing device.

In operation 1240, the oral microbiome testing system 200 may execute an operation of generating an oral health report for a pet prepared to be tested based on the health status data using the processing device.

According to an embodiment, the oral microbiome testing method 1200 may be implemented in the form of a computer program stored on a computer-readable storge medium. That is, the computer program may include instructions for implementing the oral microbiome testing method 1200, and the program instructions may be stored in a computer-readable storage medium. The computer program may include a mobile application.

According to an embodiment, the computer readable storage medium may include magnetic media such as a hard disk, a floppy disk, and a magnetic tape, optical media such as a CD-ROM or DVD, magneto-optical media such as a floptical disk, and a hardware device specially configured to store and execute computer program instructions, such as a ROM, a RAM, and a flash memory. The computer program instructions may include high-level language code that can be executed by a computer using machine code created by a compiler and an interpreter.

Although embodiments of the present invention have been described in detail above, the scope of the present invention is not limited thereto, and various modifications and improvements made by those of ordinary skill in the art using the basic concepts of the present invention described in the following claims should also be construed as being included in the scope of the present invention.

What is claimed is:

1. An oral microbiome testing system for early diagnosis of periodontal disease in a pet, comprising:
a genomic analysis device configured to generate microbiome data based on a specimen collected from the oral cavity of a pet prepared to be tested; and
a processing device configured to:
generate abundance data, prevalence data, and correlation data of the microbiome data based on a list of microorganisms of interest in a pet standard database;
generate health status data of the specimen by comparing the abundance data, prevalence data and correlation data with standard samples of the pet standard database; and
generate an oral health report for the pet prepared to be tested based on the health status data,
wherein the standard samples of the pet standard database are constructed with:
primary standard samples including stages of abundance data, prevalence data, and correlation data for a plurality of conditions including bad breath, plaque, tartar, gum inflammation, and periodontal disease; and
additional secondary standard samples including abundance data, prevalence data, and correlation data for each stage of disease progression by subdividing the disease progression stages for the primary standard samples, and
wherein the prevalence data is provided as a heatmap that visually indicates the appearance frequency of each microorganism by displaying, in different colors, the probability that a specific microorganism in the specimen exceeds a predetermined threshold in the primary and secondary standard samples.

2. The system of claim 1, wherein the genomic analysis device is configured to generate the microbiome data by identifying types of microorganisms present in the specimen through 16S rRNA sequencing based on next generation sequencing (NGS).

3. The system of claim 1, wherein the list of microorganisms of interest is configured to define n phyla and m genera referred to for the early diagnosis of periodontal disease, and
wherein the processing device is configured to generate the abundance data, prevalence data and correlation data based on the n phyla and the m genera.

4. The system of claim 3, wherein the processing device is configured to:
generate the abundance data by calculating a first compositional ratio of microorganisms present in the specimen across the n phyla and a second compositional ratio of microorganisms present in the specimen across the m genera, and
generate the health status data of the specimen by comparing the first compositional ratio and the second compositional ratio with the standard samples.

5. The system of claim 3, wherein the processing device is configured to:
generate the prevalence data by generating a first heatmap representing the probabilities that each of the n phyla will exceed a detection threshold of relative abundance and a second heatmap representing the probabilities that each of the m genera will exceed a detection threshold of relative abundance, and
generate the health status data of the specimen by comparing the first heatmap and the second heatmap with the standard samples.

6. The system of claim 3, wherein the processing device is configured to:
generate the correlation data by calculating m correlation values between m microbial populations and the representative microbial population, present in the specimen, as compared with the m genera, and
generate health status data of the specimen by comparing the m correlation values with the standard samples.

7. The system of claim 1, wherein the processing device is configured to:
determine a standard sample closest to the abundance data, prevalence data and correlation data for the specimen using a data classification model that is trained to output the standard sample closest to the input data among the standard samples, and
generate the health status data based on the standard sample determined to be closest to the specimen.

8. The system of claim 7, wherein the processing device is configured to generate the oral health report by considering metadata including breed, age, sex, body weight, oral care status, and specific diseases of the pet prepared to be tested.

9. The system of claim 1, wherein the processing device is configured to:
determine an evaluation indicator corresponding to the microbiome data based on a PCoA cluster of the pet standard database, and
generate the oral health report in further consideration of the evaluation indicator.

10. An oral microbiome testing method for early diagnosis of periodontal disease in a pet, comprising:
generating microbiome data based on a specimen collected from the oral cavity of a pet prepared to be tested using a genomic analysis device;
generating abundance data, prevalence data and correlation data of the microbiome data based on a list of microorganisms of interest in a pet standard database;
generating health status data of the specimen by comparing the abundance data, prevalence data and correlation data with standard samples of the pet standard database using the processing device; and
generating an oral health report for the pet prepared to be tested based on the health status data using the processing device,
wherein the standard samples of the pet standard database are constructed with:
primary standard samples including stages of abundance data, prevalence data, and correlation data for a plurality of conditions including bad breath, plaque, tartar, gum inflammation, and periodontal disease; and
additional secondary standard samples including abundance data, prevalence data, and correlation data for each stage of disease progression by subdividing the disease progression stages for the primary standard samples, and
wherein the prevalence data is provided as a heatmap that visually indicates the appearance frequency of each microorganism by displaying, in different colors, the probability that a specific microorganism in the specimen exceeds a predetermined threshold in the primary and secondary standard samples.
